(19) United States
(12) Patent Application Publication
(10) Pub. No.: US 2016/0169892 A1
(43) Pub. Date: Jun. 16, 2016

(54) MONOCLONAL ANTIBODIES, HYBRIDOMA CELL LINES, METHODS AND ASSAYS FOR DETECTING FUNGAL PHYTASE

(71) Applicants: Hong Ke, Burnaby (CA); Jun Zhang, Burnaby (CA); Jian Zheng, Jinan (CN); Yuhua Zang, Jinan (CN); Qi Liu, Beijing (CN)

(72) Inventors: Hong Ke, Burnaby (CA); Jun Zhang, Burnaby (CA); Jian Zheng, Jinan (CN); Yuhua Zang, Jinan (CN); Qi Liu, Beijing (CN)

(21) Appl. No.: 14/566,240

(22) Filed: Dec. 10, 2014

(51) Int. Cl. G01N 33/573; C07K 16/40

(52) U.S. Cl. CPC G01N 33/573; C07K 16/40; G01N 2333/916; C07K 2317/14; C07K 2317/33

(57) ABSTRACT

This invention relates to the field of immunology and more specifically relates to antiphytase monoclonal antibodies and immunoassay methods for the detection of a phytase from or derived from Aspergillus niger (phyA2) phytase, in particular, EH10a, FA7, AF9a and CC1 antiphytase antibodies. The invention further relates to hybridoma cell lines that produce antiphytase monoclonal antibodies.

### FIELD OF THE INVENTION

This invention relates to the field of immunology and more specifically to monoclonal antibodies, immunoassay methods, including ELISA and immunostrip assays, for detection of phytase, specifically Aspergillus niger (phyA2) derived phytase, in genetically modified organisms, such as corn. The invention includes monoclonal antibodies capable of detecting glycosylated phytase. The invention further includes hybridoma cell lines that produce anti-phytase monoclonal antibodies and its application in various detection methods and assays.

### BACKGROUND OF INVENTION

Phytase (myo-inositol hexakisphosphate phosphohydrolase) EC 3.1.3.8 is part a class of phosphatases which can catalyze the sequential hydrolysis of phytate to lower phosphorylated inositol and inorganic phosphates. Phytate is the main storage form of phosphorus in livestock feed such as seeds and cereal grains, representing nearly 90% of their total phosphorus content. The digestive microbial fauna of monogastric animals lack the necessary phosphorus hydrolyzing enzymes and as a result much undigested phytate-associated phosphorus is lost into the environment. This can lead to excessive phosphorus loading in soil and water and the ensuing pollution can affect other ecosystems. Inorganic phosphorus or phytase as feed supplements and the subsequent development and application of transgenic phytase plants has been shown as possible solutions to greatly improve the phytate antinutrient factor, prompting the search for more temperature and pH tolerant phytases and propelled phytase optimization technology through genetic and protein engineering.

This has precipitated a growing need for accurate verification and increased awareness regarding the distribution of genetically modified phytase organisms and products, particularly those pertaining to agriculture and in the development of food nutritional and environmental management strategies wherein concerns of phytate mineral availability and environmental issues need to be assessed. Development of a rapid diagnostic test (RDT), which is a qualitative immunoassay (consisting of target specific antibodies to phytase) used in point-of-care testing, for transgenic A. niger (phyA2) phytase would offer an efficient and convenient method of detection.

Phytases can be glycosylated and the level of glycosylation is known to be highly variable, between different expression systems and individuals within a given expression system. Glycosylation can have many effects on the properties of a protein, such as on stability, solubility, and metabolic energy. Glycosylation of phytase have been shown to increase thermostability which would be an invaluable feature when there are concerns regarding enzyme activity loss due to heat such as that from feed pelleting. Phytases for commercial use have been isolated mainly from fungi and bacteria and selection for efficacious products is greatly dependent on the source, tolerance to processing factors, digestive resistance and production costs. Aspergillus niger phytase (phyA2) which has 10 potential glycosylation sites was cloned and expressed in a methylotrophic yeast, Pichia pastoris. Phytase expressed from yeast is known to be glycosylated and the said yeast-expressed A. niger phytase had facilitated the invention to include the ability to detect glycosylated phytase.

The present invention can fulfil the need for a rapid diagnostic test for an efficient detection of phytase by production of anti-phytase monoclonal antibodies, of which the phytase antigen may be glycosylated, hybridoma cell lines, and the construction of immunological assays that would deliver immediate results, requiring little skill or additional equipment.

### SUMMARY OF THE INVENTION

The immunoassay for detecting for the presence of phytase in a sample is supplied. This would include the monoclonal antibodies which can detect a phytase, specifically an A. niger (phyA2) phytase that resulted in the production of EH10a, FA7, AF9a and CC1. Phytase can be found in various microorganisms, fungi and plants. The invention can be used to detect the phytase protein in genetically modified (GM) plants which encode the transgenic phytase gene. The genetically modified phytase plants can be used for human and non-human consumption in the form of agricultural plants or plant by-products.

The phytase (phyA2) protein may be purified from A. niger and grown in the methylotrophic yeast, Pichia pastoris. This protein is then introduced into animals to produce polyclonal or monoclonal antibodies.

The monoclonal antibodies have a high specificity and sensitivity for phytase that is distinguishable from plant- and yeast-expressed phytase and can be applied to immunoassay methods for detection of phytase in genetically modified organisms. The specificity of the phytase monoclonal antibody pairs EH10a-FA7 and AF91-CC1 (where one of the pair is immobilized on the surface of the capture membrane and the other conjugated to gold particles near the sample pad) in an antibody-coated lateral test strip were constructed and tested with different seed varieties of commercial plants. Phytase could not be detected in any of the non-GM phytase seed varieties tested using either of the antibody pairs. Detection limit of the immunolateral test strip to recombinant phytase using the EH10a-FA7 antibody pair was 5 ng/ml whereas the detection limit to GM phytase corn using the AF9a-CC1 antibody pair was as low as 2 ng/ml. The strips were confirmed to be stable when kept at RT for at least 1 year.

Analyses of the protein detected from the plant- and yeast-expressed phytase showed that different sizes were detected. Monoclonal antibody pairs EH10a and FA7 which had detected the larger-sized protein may be able to recognize glycosylated phytase. This suggests that the epitope binding sites of one or both of the monoclonal antibody pairs EH10a and FA7 may consist of a carbohydrate moiety. Correct detection of glycosylation within transgenic phytase plants may be essential when a marketable product with a more thermostable enzyme is required. A rapid diagnostic test in the form of an immunochromatographic capillary flow assay consisting of specific and sensitive monoclonal antibodies allows for an efficient and sensitive point-of-care testing method that would deliver immediate results, requiring little skill or additional equipment. Here we present the development of such a said rapid diagnostic test that is able to detect phytase from genetically modified crops.

### BRIEF DESCRIPTION OF THE FIGURES

FIGS. 1A and B show that the monoclonal antibodies target phytase. FIG. 1A shows that all seed samples tested contains protein using a 15% polyacrylamide protein gel. Fifty micrograms of plant seeds were ground in 500 μl Laemmli buffer of which 20 μl were loaded into each lane. The lanes are as follows: (1) water, (2) 10 mM Tris-HCl, pH 8.0, (3) Marker, (4) 1 μg recombinant phytase, (5) GM phytase corn, (6) corn 1, (7) green bean, (8) white bean, (9) Pickseed2733 corn, (10) WCS F1 corn, (11) GM corn 1, (12) GM corn 2, (13) GM corn 3, (14) GM corn 4 and (15) GM soybean. FIG. 1B show that Western blot analyses were conducted on the samples used in (A), except that 5 μg of recombinant phytase and 100 mg of ground plant seeds were used and probed with an equal mixture of monoclonal antibodies AF9a, CC1, EH10a and FA7. The protein marker sizes kilodaltons (kD) are indicated on the left of the blot. GM=genetically modified; WCS=West Coast Seeds. Unless stipulated, GM corn samples are not GM phytase corn.

FIGS. 2A and B show that both match pair EH10a-FA7 and AF9a-CC1 antibodies show high specificity to phytase using lateral flow test strips. FIG. 2A show that phytase specificity analysis was conducted using lateral flow strips containing the EH10a gold-conjugated antibody and the FA7 capture membrane antibody. Strips 1-4 were immersed in a 10 ml solution consisting of (1) 200 μg/ml recombinant phytase, (2) 200 μg/ml transgenic phytase corn, (3) 10 mM Tris-HCl and (4) distilled water. Strips 5-28 were immersed in the supernatant of 2 g of seeds ground in 10 ml of 10 mM Tris-HCl for 1-5 minutes. The strips were tested using the following seeds: (5) corn 1, (6) corn 2, (7) corn 3, (8) corn 4, (9) GM corn 1, (10) GM soybean, (11) Pickseed2733 corn, (12) GM corn 2, (13) GM corn 3, (14) GM corn 4, (15) WCS F1 corn 1, (16) WCS F1 corn 2, (17) WCS P corn 1, (18) WCS P corn 2, (19) WCS DF1 corn, (20) green soybean, (21) WCS corn, (22) green bean, (23) white bean, (24) corn 5, (25) corn 6, (26) corn 7, (27) GM corn 5 and (28) GM corn 6. For each strip the upper line is the control (C) line containing antibodies to goat anti-mouse IgG and the lower line (if present) is the test (T) line with the FA7 capture antibody. The corn used in strips 5 to 8 and 24 to 26 were generic (non-GM) varieties. FIG. 2B show that phytase specificity analysis was conducted using lateral flow strips containing the AF9a gold-conjugated antibody and the CC1 capture membrane antibody. Each strip was prepared and tested with the same samples as described in FIG. 2A except the lower T line (if present) contains the CC1 capture antibody.

FIGS. 3A and B show that both match pair EH10a-FA7 and AF9a-CC1 antibodies show high sensitivity to phytase using lateral flow test strips. FIG. 3A shows that phytase sensitivity analysis of the EH10a-FA7 antibodies was conducted, using concentrations from 0.002 to 200 μg/ml of recombinant phytase applied to lateral flow strips containing the EH10a gold-conjugated antibody and the FA7 capture membrane antibody. FIG. 3B shows that phytase sensitivity analysis of the AF9a-CC1 antibodies was conducted, using concentrations from 0.001 to 400 μg/ml of the GM phytase corn applied to lateral flow strips containing the AF9a gold-conjugated antibody and the CC1 capture membrane antibody. The strips were immersed in a 10 ml solution of recombinant phytase in FIG. 3A and GM phytase corn in FIG. 3B in the various concentrations indicated above the strips. The arrangement of the control (C) and test (T) lines along the strips are as described in FIG. 2.

FIGS. 4A and B reveal the nature of phytase epitopes using the monoclonal antibodies match-pair EH10a-FA7 and AF9a-CC1 over time. FIG. 4A indicates that 10 μl of (200 μg/ml) purified recombinant phytase (phy2A) expressed from yeast (top row) and (200 μg/ml) phytase expressed from GM corn (bottom row) were prepared and evaluated every 2 weeks over a period of 10 weeks using western blot analysis. Each blot was probed with a mixture of monoclonal antibodies EH10a, FA7, AF9a and CC1 (in equal 1:400 concentrations). Record dates are indicated above the blot with the protein marker sizes kilodaltons (kD) to the left. FIG. 4B shows that purified recombinant phytase (phy2A) expressed from yeast (top row) and phytase expressed from GM corn (bottom row) were prepared and tested along immunolateral flow strips. Test strips containing the EH10a gold-conjugated antibody and the FA7 capture membrane antibody (green coloured) and AF9a conjugate antibody and the CC1 capture antibody (brown coloured) were immersed in a solution of the prepared phytase and detection was recorded on the dates indicated above the strips. The arrangement of the control (C) and test (T) lines along the strips are as described in FIG. 2.

### DETAILED DESCRIPTION OF THE INVENTION EMBODIMENTS

Anti-phytase monoclonal antibodies, hybridomas, and the immunoassays, for the detection of phytase in a sample are described.

The methodology of the invention may be used to detect enzymes in samples, such as those in agricultural crops and food by-products. Many enzymes have been detected in such a manner by those experienced in the art. Specificity is important in the proper construction of immunoassays to detect phytase in genetically modified organisms and their products. Therefore, to ensure the manufacture of successful commercial products, highly specific monoclonal antibodies to phytase were developed. Described is an immunoassay which employs a test kit strip format and consists of sensitive and specific monoclonal antibodies for the detection of phytase in genetically modified organisms, such as those in agricultural products.

### Recombinant Phytase Protein

Preparation of antigenic recombinant protein, such as Aspergillus niger phytase, was cloned into an appropriate DNA vehicle with a suitable promoter, transformed into an suitable host strain, e.g., a bacterial, insect or yeast host, such as Pichia pastoris, by means of heat or chemical inducement whereby cells are incubated until sufficient concentrations are reached after which cells are cultured for an additional period to yield recombinant enzyme protein. The protein is subsequently purified from pelleted cells that were subjected to physical or chemical disruption by methods known to those skilled in the art.

### Antibodies

The antibodies produced in this invention may be made using a rabbit, chicken, mouse or a goat. For example, mice were immunized with multiple subcutaneous or intraperitoneal injections of recombinant phytase over a set period. The immunization protocol can be selected by one skilled in the art. Each mouse was immunized with a mixture of the recombinant phytase and an immunizing agent, such as complete Freund's adjuvant. Subsequent booster injections were given with another immunizing agent, such as incomplete Freund's adjuvant, over a set period. After which the immune response was assessed by measuring polyclonal antibody titer in immunized animal sera using indirect ELISA. Such techniques are known to those skilled in the art. Immunized mice with the highest titers are selected for hybridoma production and given a final booster injection before their spleen cells were to be harvested. The harvested spleen cells were fused with myeloma cells, usually of mouse or rat origin, producing hybridoma cells that are suspended in an enriched medium, such as RPMI 1640.

### Hybridoma Cell Lines

The hybridoma cells were seeded in tissue culture plates in a suitable medium, such as hypoxanthine-aminopterin-thymidine (HAT), which contains one or more substances that inhibit the growth or survival of the unfused, immortalized cells. Cell lines which showed the strongest positive signals were selected for using indirect ELISA and processed to maximize monoclonality and stability. Supernatants from these clones were retested using indirect ELISA and positive candidates were selected for large scale production in a nonselective medium and stored in liquid nitrogen until required.

The hybridoma cell lines are assigned as EH10a, FA7, AF9a and CC1.

### Monoclonal Antibodies

The anti-phytase antibodies were monoclonal antibodies. Monoclonal antibodies raised against proteins, such as the recombinant A. niger phytase, were produced using a standard ascitic fluid method as described in the EXAMPLE below. The production and purification protocol can be selected by one skilled in the art. The said hybridomas produced in this invention may involve a mouse, hamster, or other appropriate host animal, which is typically immunized with an immunizing agent to elicit lymphocytes that produce or are capable of producing monoclonal antibodies that will specifically bind to the said immunizing agent. For example, mice could be injected intraperitoneally with the said hybridoma cells for a set time. The ascitic fluid would be drained and purified, for example, by a protein A affinity chromatography, protein A-Sepharose, hydroxylapatite chromatography, gel electrophoresis, dialysis, or affinity chromatography, method to obtain high quality monoclonal antibodies. The titer was determined by methods known to those skilled in the art, for example, indirect ELISA.

Monoclonal antibodies of the invention using the hybridoma cell lines are as follows:

Cell culture line EH10a deposited as EH10a
Cell culture line FA7 deposited as FA7
Cell culture line AF9a deposited as AF9a
Cell culture line CC1 deposited as CC1

### Immunoassay

The antibodies described above may be used in a various assays to determine the presence of the phytase in a sample. The antibodies may be used in any quantitative or qualitative immunoassay.

A typical quantitative in entail the following steps: a phytase-containing sample, for example from a genetically modified corn seed, is captured onto a solid phase using a primary antibody. In one embodiment, the primary antibody is a mouse anti-phytase antibody, coated onto the solid carrier. A secondary anti-phytase antibody is further added. In one embodiment, the secondary antibody is labelled or unlabelled goat anti-phytase antibody. After washing to remove unbound antibody, the label on the bound secondary antibody is detected. In one embodiment, the label is horse radish peroxidise (HRP). A substrate to detect the label is added and colour development is measured by reading the absorbance.

A typical protocol entails:

1. Coat and incubate a solid carrier, such as wells in a 96-well plate, with primary anti-phytase antibody.
2. Wash the carrier to remove unbound with primary anti-phytase antibody
3. Prepare the phytase-containing sample and apply to prepared solid carrier
4. Wash the carrier to remove unbound sample
5. Apply labelled secondary anti-phytase antibody which will bind to the sample
6. Wash the carrier to remove unbound secondary anti-phytase antibody
7. Add a substrate which binds to the label of the secondary anti-phytase antibody to form primary anti-phytase antibody-phytase sample-secondary anti-phytase antibody complex
8. Measure the amount of labelled secondary anti-phytase antibody

In one embodiment, the phytase is a fungal phytase, more particularly, an Aspergillus niger phytase.

The antibodies can be used a qualitative immunoassay for the detection of a transgenic enzyme, such phytase in genetically modified organisms. This invention utilizes a test strip immunoassay whereby antibodies are coated on a membrane attached to a solid support strip. As a liquid sample (or solid sample mixed in a liquid, such as water) is placed on the sample pad at one end of the strip, it will be drawn up by capillary action and migrates toward the distal end of the strip. In one embodiment if the antigen within the sample reacts with the antibodies that are labelled directly with a detectable label for identification and visualization of the antigen, such as phytase protein, in the sample pad and further reacts with antibodies, also anti-antigenic, on the capture line on the solid membrane backing as it moves the length of the strip, a positive signal will be detected on the said capture line. Labels for use in immunoassays are generally known to those skilled in the art and include, but are not limited to enzymes, radioisotopes, paramagnetic nanoparticles, fluorescent, luminescent, and chromogenic substances including colored particles such as colloidal gold and latex beads. In a preferred embodiment, colloidal gold is the visualization particle means of detection. Methods of labelling antibodies and assay conjugates are well known to those skilled in the art.

In one embodiment the phytase is a fungal phytase. In a more particular embodiment, the phytase (phyA2) is from Aspergillus niger. In another embodiment, the phytase is a transgenic protein found in genetically modified organism, such as corn. In other embodiments, the solid membrane backing is usually made up of cellulose acetate, cellulose, nitrocellulose or nylon. In a preferred embodiment, the solid phase format is nitrocellulose. In another preferred embodiment, the solid support strip further comprises a sample absorption pad at one end. In a more preferred embodiment, the immunoassay further comprises a strip comprising a labelled anti-phytase antibody at the sample absorption pad end and a distal wicking pad to draw the liquid forward at the other end. Methods for coupling antibodies to solid phases are known to those skilled in the art.

A highly sensitive immunoassay employing the antibodies described above is provided. The assay is useful for detection of phytase protein in genetically modified organisms that have been engineered to include a gene encoding a phytase gene. The immunoassay is capable of detecting low concentrations of the protein in samples, such as in genetically modified crop samples. As described above, the antibodies are highly specific and sensitive as to react with epitopes on the phytase protein, thus providing for an accurate determination of the presence of the phytase protein in a genetically modified phytase organism, such as corn.

The sample may be obtained from any portion of any genetically modified phytase organism, for example, the sample may be any plant tissue or extract including root, stem, stalk, leaf, or seed or products derived from such crops, such as food products.

The least amount of reaction time that results in binding of the phytase to the antibodies is desired to minimize the time required to complete the assay. An appropriate reaction time period for an immunoassay test strip is between one second and ten minutes. A reaction time of less than five minutes is preferred.

### DESCRIPTION OF THE PREFERRED EMBODIMENT

The immunoassay methods described above will be further understood with reference to the following, but not limited to, examples. The examples below show typical experimental protocols and reagents that can be used in the detection of phytase in samples such plants or plant materials. It should be understood; however, that the detailed description and the specific examples, while indicating preferred embodiments of the invention, are given by way of illustration only, and any change and/or modification of the invention will be at the discretion of those skilled in the art from these detailed descriptions and examples.

### EXAMPLES

These methods and materials describe the general procedure for preparing the corn seed samples for testing and the production of the polyclonal and monoclonal antibodies used in the examples described below.

### Materials and Methods

Corn Sample: The corn seed protein was genetically modified phytase corn seed sample. Corn kernels were ground in a blender. The resulting corn flour was suspended in 5 ml distilled water to solubilize the proteins. The supernatant was tested in either the ELISA or with the immunoassay test strips.
Recombinant phytase protein: The antigenic Aspergillus niger recombinant phytase protein was cloned into a DNA plasmid with a suitable promoter and transformed into the Pichia pastoris yeast host.

### Production of Monoclonal Antibodies

Female mice were immunized with four intraperitoneal injections of recombinant phytase (expressed from yeast Pichia pastoris) over a period of 2 weeks. Prior to immunization, blood was collected from the inner canthus of mice to be used as a negative control. Each mouse was immunized with 1:1 mixture (v/v) of the recombinant phytase and complete Freund's adjuvant. After the two weeks, three separate booster injections were given in the same proportion of immunogen emulsified using incomplete Freund's adjuvant over a period of 2 weeks. A second round of booster injections was given over an additional 2 weeks, after which the immune response was assessed by measuring the titer of polyclonal antibody in mice sera using indirect ELISA. Immunized mice with the highest titers were selected for hybridoma production and given a final booster injection via the tail vein 3 days before their spleen cells were to be harvested and used just prior to cell fusion. The other sera were pooled and used as a positive control.

Three days after the last intravenous booster injection, the immunized mice were eye-bled (to verify high antibody production), had splenocytes removed and were subsequently euthanized. The harvested spleen cells were fused with myeloma cells and the resulting hybridoma cells were suspended in enriched RPMI 1640 media. The cells were centrifuged at 500 g for 10 min and the subsequent pelleted cells were suspended in HAT media and incubated for 2 weeks. Cells were then seeded into 96-well tissue culture plates and kept in a hypoxanthine thymidine media for a further 2 weeks. Using indirect ELISA, cell lines which showed the strongest positive signals were recloned three times by limiting dilution using spleen cells from non-immunized mice to maximize monoclonality and stability. Supernatants from these clones were retested using indirect ELISA and positive candidates were selected for large scale production in a nonselective medium and stored in liquid nitrogen until required.

Monoclonal antibodies (MAbs) raised against the said recombinant A. niger phyA2 phytase were produced using a standard ascitic fluid method. Each mouse, primed with liquid paraffin, was injected intraperitoneally with 1×10⁶ hybridoma cells. One to two weeks later, the ascitic fluid was drained and centrifuged at 4000 rpm at 4° C. for 10 min. The collected supernatants were precipitated in 50% saturated ammonium sulfate (pH 7.4), followed by extensive dialysis with 0.02 M phosphate solution (pH 7.4) at 4° C. The solution was purified by protein A affinity chromatography to obtain high quality MAbs. The flow through was collected in 4-5 ml fractions whereby the OD₂₈₀ of each fractions was monitored until the reading dropped below 0.05 to ensure that there was no more unbound protein in the solution. The column was then eluted by loading 5 ml, 1 ml at a time, of elution buffer (pH 3.0). To neutralize the pH, the eluants were collected in tubes containing 300 μl of 1 M Tris-HCl (pH 9.0). Eluant fractions of 1 ml were collected and monitored until an OD₂₈₀ reading of 0.05 was reached.

The purity of the eluted products was assessed by 10% SDS-PAGE. The titer of MAbs was determined by indirect ELISA. The extensive screening process yielded MAbs which showed the highest detection response for large scale production.

### Example 1

### Phytase Indirect ELISA

This example describes the detection and quantitative measurement of phytase antigen in culture supernatant samples using the enzyme-linked immunosorbent assay (ELISA) immunological technique.

### Procedure

Each well of various 96-well microplates was coated with 100 μl of (yeast expressed) recombinant phytase antigens, which included positive and negative controls, in 0.1 M NaHCO₃ at a concentration of 10 μg/ml and incubated overnight at 4° C. After blocking for 2 h with 1×PBS and 1% BSA, 100 μl of hybridoma culture supernatants, immunized mouse serum (positive control) and SP2/0 (negative control) were added to respective wells and incubated at 37° C. for 1 h. Plates were washed three times with PBST and each well was incubated with 100 μl horseradish peroxidise conjugated goat anti-mouse immunoglobulin (IgG-HRP) in blocking buffer (at 1:1000) for 30 min at 37° C. Finally the plates were washed five times with 1×PBST and developed with 3,3′,5,5′-tetramethylbenzidine (TMB) liquid substrate system for ELISA. The reaction was terminated by supplementing per well with 50 μl of 1 M sulfuric acid (H₂SO₄). The absorbance values of the wells from the ELISA were recorded at 450 nm. The titer of the antibody preparation was defined as the highest dilution that could give a reading of 0.05. One indirect ELISA unit was defined as the smallest amount of antibody which can detect a positive antigen signal.

The titer of the antibodies in the supernatant culture of hybridomas and ascites indicated high activity (all >10⁻⁶).

### Example 2

### ELISA to Characterize Anti-Phytase Epitopes (Antibody Binding Sites)

This example describes the quantitative measurement of the epitopes of the purified MAbs to phytase characterized by ELISA and the additivity index (AI) described by Friguet et al. [(J. Immunol. Methods, 60:351(1983)].

### Procedure

The wells of a 96-well plate were coated with 100 μl of 2 μg/ml (yeast expressed) recombinant phytase and incubated overnight at 4° C. The following day, the wells were blocked then incubated with 100 μl of antibodies, EH10a, FA7, AF9a, CC1 individually or in paired combinations (50 μl each) of equivalent concentrations at 1:1000 overnight at 4° C. For each treatment there were three replicate wells. The following day, the wells were incubated with 100 μl of a goat anti-mouse IgG-HRP secondary antibody at 1:1000 for 30 min at 37° C. The wells were developed and the reaction terminated by addition of an equal volume of 1 M H₂SO₄. Similar treatment sample wells were combined and the absorbance value for each treatment was recorded at 450 nm. The AI was calculated using the following equation: {[2A₁₊₂/(A₁+A₂)]−1}×100%, where A₁, A₂ and A₁₊₂ are the absorbance values for the individual antibodies and the respective combined pairs. If the two antibodies are directed against different epitopes (no competition), A₁₊₂ should be equal to the sum of A₁ and A₂ and the AI value should approach 100%. If the two antibodies are directed against the same epitope (competition), A₁₊₂ should be equal to the mean value for A₁ and A₂ and AI should be close to 0%. The threshold in this study was determined by AI≧40%.

TABLE 1
AI values of the epitopes of the monoclonal antibodies to phytase

| Antibody pairs | Additive Index (AI) (%) |
|---|---|
| FA7 + AF9a | 67.7 |
| FA7 + CC1 | 91.4 |
| AF9a + EH10a | 57.7 |
| EH10a + CC1 | 69.7 |
| FA7 + EH10a | 59.6 |
| AF9a + CC1 | ≧99 |

As shown in Table 1, the AI data indicate that if any pair of these monoclonal antibodies would result in the targeting of a different phytase epitope.

### Example 3

### Detection of Phytase Protein in Plant Seeds

This example describes the detection of phytase protein in plant seeds using western blot analysis and anti-phytase monoclonal antibodies (EH10a, FA7, AF9a and CC1).

### Procedure

Protein and western blot analysis on the specificity of the MAbs was evaluated by 15% SDS-PAGE. Sodium dodecyl sulfate-polyacrylamide gel or SDS-PAGE was prepared as a two layered gel whereby the lower, resolving gel layer consists of 15% acrylamide/bis-acrylamide, 390 mM Tris, pH 8.8, 0.1% SDS (w/v), 0.1% ammonium persulfate (w/v) and 0.1% TEMED and the upper, stacking gel consists of 4% acrylamide/bis-acrylamide, 125 mM Tris, pH 6.8, 0.1% (w/v) SDS, 0.1% (w/v) ammonium persulfate and 0.1% tetramethylethylenediamine (TEMED). The upper, stacking gel was prepared to accommodate a 15-well sample loading comb.

Samples evaluated consisted of 1 μg of (yeast expressed) recombinant phytase and 50 mg of ground seeds from genetically modified phytase corn, generic corn, green bean, white bean, Pickseed2733 corn, WCS F1 corn, four varieties of GM corn and one GM soybean variety. The phytase was released from the genetically modified phytase corn by homogenizing the corn in a modified Tris buffer [50 mM Tris-HCl (pH 8.0), 10 mM KCl, 3 mM MgCl2, 1 mM EDTA, 1 mM β-mercaptoethanol, 0.1% BSA, 13% sucrose and SigmaFAST™ Protease inhibitor cocktail (Sigma-Aldrich, St. Louis, Mo.) at 400 μl per 100 mg tissue] after which the sample was centrifuged at 4500 g for 10 min. Laemmli buffer was then added to the supernatant and incubated at 65° C. for 20 min. The remaining seeds were ground were homogenized in 500 μl of Laemmli buffer, boiled for 10 min, and centrifuged for 2 min at 12,000 g. To improved western blot detection, 5 μg of recombinant phytase and 100 mg of ground seeds were used. Twenty microlitres of each prepared seed supernatant was used for protein and western blot analysis. The recombinant phytase, 20 μl of water, and 10 mM Tris (the latter two serving as negative controls) were also boiled in Laemmli buffer as described above.

Twenty microlitres of each supernatant sample is loaded onto the stacking gel into separate wells. The gels were run at a voltage of 200 V for 45 min in a running buffer consisting of 25 mM Tris, 200 mM glycine and 0.1% (w/v) SDS. For the purpose of evaluating total protein, SDS-PAGEs are stained with Coomassie Brilliant Blue stain [9.375% (w/v) trichloroacetic acid and 0.0625% (w/v) Brilliant Blue stain] whereas SDS-PAGEs for western blot analysis are transferred to a nitrocellulose membrane using semi-dry transfer blot apparatus running at a voltage of 20 V for 2 h with a transfer buffer consisting of 25 mM Tris, 192 mM glycine and 20% (w/v) methanol. After which the blot is blocked in 5% milk power and 1×PBST (phosphate buffered saline-Tween 20) or 1×TBST (tris buffered saline-Tween 20) for 1 h and probed with a combined mixture of the monoclonal antibodies (EH10a, FA7, AF9a, and CC1) at 1:400 each] overnight. The following day the blot is washed with 1×PBST or 1×TBST and incubated with horseradish peroxidise conjugated goat anti-mouse immunoglobulin (IgG-HRP at 1:1000) for 90 min at RT. Proteins on the blot were developed using the DAB (3,3′-diaminobenzidine tetrahydrochloride; Sigma-Aldrich, St. Louis, Mo.) method with 0.1% hydrogen peroxide.

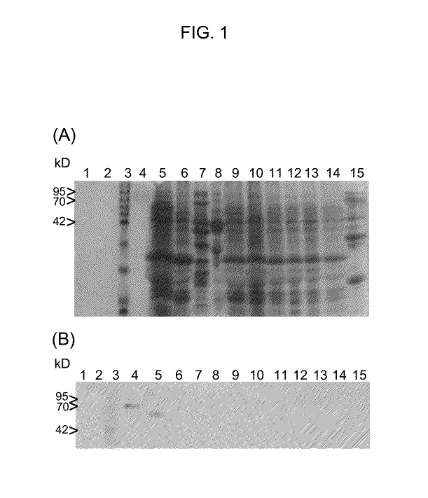

As shown in FIG. 1, the results indicate that the monoclonal antibodies (EH10a, FA7, AF9a and CC1) were able to only detect protein phytase from the recombinant phytase protein and GM phytase corn but none from the non-GM seed varieties. The size of the detected phytase protein is of different between the (yeast expressed) recombinant protein and GM corn.

### Example 4

### Anti-Phytase Immunoassay Test Strips (A)

This example describes the use of immunoassay strips to test the specificity of anti-phytase antibodies by comparison with seeds from other plant varieties.

### Procedure

Seeds from various plants [six varieties of genetically modified corn, one variety of a genetically modified soybean, corn (2733) from Pickseed, six corn varieties from West Coast Seeds (=WCS; two of F1, two of P, one of DF1 and one unknown), green bean, white bean and seven generic varieties of corn (purchased from local markets)]. Two grams of each seed variety was ground in 10 mM Tris-HCl. The absorption pad end of a prepared test strip was immersed in seed supernatant. In addition, test strips were also immersed in 200 μg/ml of (yeast expressed) recombinant phytase, 200 μg/ml of transgenic corn, distilled water and 10 mM Tris-HCl (the latter two serving as negative controls). A response was observed after 1-5 min. Two bands that appeared at both the test and control site represent a positive test result. Only one band at the control site represents a negative test result. The absence of a line at the control site indicates the test is invalid.

The phytase lateral flow test strips consisted of a sample binding area called an analyte absorption pad, followed by a conjugate pad, a nitrocellulose membrane and a terminal wicking pad. The detection phytase antibody (the test line antibody consisting of either FA7 or CC1) and the goat anti-mouse IgG (the control line antibody, placed in parallel above the test line antibody) were diluted to a standard concentration of 1.5 mg/ml with 10 mM Tris-HCl (pH 8.0) and applied in a thin line onto a nitrocellulose membrane, allowed to dry for 2 h, then blocked with 5% milk powder and dried at 37° C. for 24 h. The colloidal gold conjugated phytase capture antibody was prepared using 100 ml of 0.01% (w/v) chloroauric acid (HAuCl₄) in a 250 ml siliconized flask which was heated to boiling in a microwave oven. After which 1.4 ml 1% trisodium citrate was added. After the colloidal gold solution was allowed to cool gradually, the pH was adjusted to 8.4 with 1% (w/v) potassium carbonate. Colloidal gold to be conjugated with either the EH10a or AF9a antibody was prepared individually by adding the antibody dropwise into 10 ml of colloidal gold solution while being stirred for 30 min using a magnetic stirrer. After the solution was stabilized at 4° C. for 30 min, 1 ml of 10% (w/v) bovine serum albumin (BSA) was added to block access reactivity of the gold colloid. The mixture was then stirred for an additional 30 min and incubated at 4° C. for 2 h. After which the mixture was centrifuged at 3000 g for 4° C. for 30 min; the supernatant was further centrifuged at 14,000 g at 4° C. for 45 min and the resulting conjugate pellet was suspended in 10 mM borax buffer (pH 8.0) containing 2% (w/v) BSA and 0.05% sodium azide (NaN₃). The prepared conjugate phytase antibody (either EH10a or AF9a) was sprayed twice onto fibreglass (0.5-1.5 cm×25 cm) and dried at 37° C. The optimal concentration of the conjugate antibody is with an OD of 50.

The components were assembled as a unit wherein the phytase capture membrane was placed on the solid support plastic backing board with the phytase antibody capture line exposed in the middle with the goat anti-mouse IgG control line in parallel above the phytase antibody capture line. The gold-conjugated phytase antibody is placed ahead of the absorbent sample pad which is at one end and the wicking pad at the other end. The monoclonal antibody pairs (conjugate-capture) EH10a-FA7 and AF9a-CC1 were prepared on respective strip tests. The assembled unit was then sliced into 4-mm wide strips.

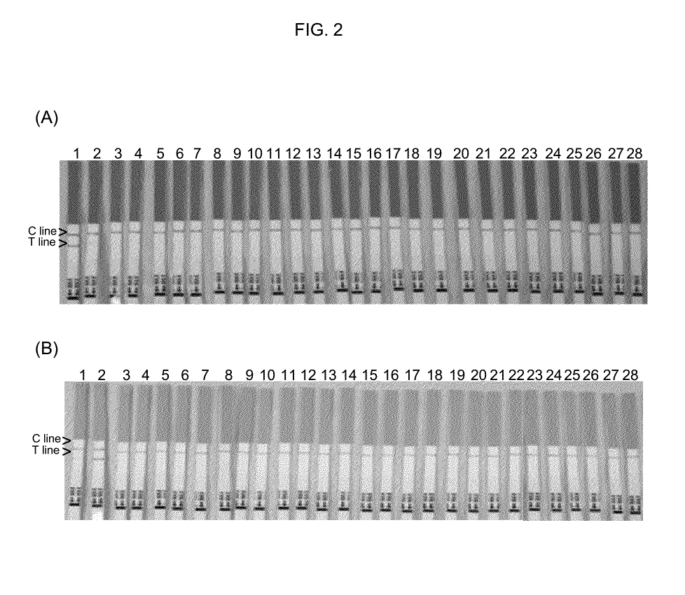

As shown in FIG. 2, the results indicate that the monoclonal antibody match pairs EH10a-FA7 and AF9a-CC1 was able to detect the (yeast expressed) recombinant phytase and the GM corn phytase, respectively, but none of the non-GM seed varieties.

### Example 5

### Anti-Phytase Immunoassay Test Strips (B)

This example describes the use of immunoassay strips to test the sensitivity of the anti-phytase antibodies (AF9a, CC1, EH10a and FA7) to phytase samples.

In photos which illustrate embodiments of the invention, FIG. 2 reveals the sensitivity of the antibodies to phytase. Immunolateral flow test strips were prepared by testing the (yeast expressed) recombinant phytase diluted in concentrations from 200 to 0.002 μg/ml by 10 mM Tris-HCl (pH 8.0) using the EH10a-FA7 MAb match pairs whereas the GM phytase corn was tested in concentrations from 400 to 0.001 μg/ml using the AF9a-CC1 match pairs. The immunolateral flow test strips were assembled and processed in a manner as described above in which the recombinant phytase and GM phytase corn samples in the said concentrations were tested and evaluated as described above.

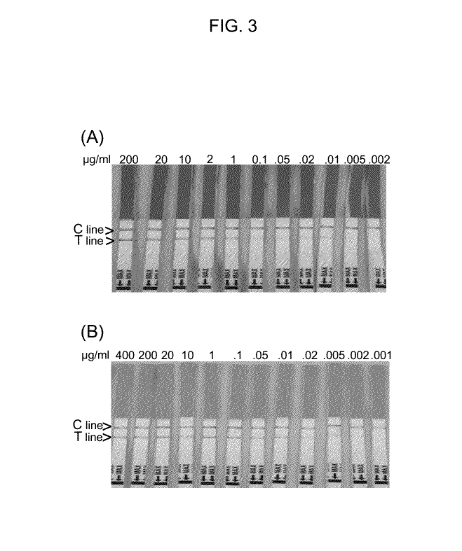

As shown in FIG. 3, the results indicate that the monoclonal antibody match pairs EH10a-FA7 and AF9a-CC1 were able to detect concentrations as low as 5 ng of the (yeast expressed) recombinant phytase and as low as 2 ng of the GM corn phytase, respectively.

### Example 6

### Role of Glycosylation in Anti-Phytase Antibody Epitopes

This example characterizes the role of glycosylation in anti-phytase antibody epitopes using western blot analysis and phytase immunoassay test strips with the anti-phytase monoclonal antibodies (EH10a, FA7, AF9a and CC1).

### Procedure

To evaluate the role of glycosylation in the MAbs epitope binding sites for (yeast expressed) recombinant phytase and the GM corn phytase, an additional western blot analysis was performed. Ground GM phytase corn was reconstituted to a concentration of 200 μg/ml and stored at 4° C. until required. Every 2 weeks for a period of 10 weeks, 10 μl from each of the recombinant phytase and GM phytase corn was boiled with 10 μl of loading buffer for 5 min and loaded onto a 10% SDS-PAGE. The SDS-PAGE and western blot were prepared as described above. Each transferred membrane blot was incubated overnight at 4° C. with a combined mixture of MAbs (EH10a, FA7, AF9a, and CC1) as described above. The subsequent steps are as described above, except the concentration of the IgG-HRP secondary antibody was 1:5000 with an incubation time of 30 min.

The role of glycosylation on the MAbs epitope binding sites for (yeast-expressed) recombinant phytase and the GM corn phytase was evaluated along immunolateral flow test strips. Individual test strips consisting of either the EH10a-FA7 or the AF9a-CC1 MAb match pairs were prepared as described above. The samples were stored at 4° C. until required. The prepared strips were immersed in the reconstituted recombinant or GM corn phytase samples every 2 weeks for a period of 10 weeks. With the “MAX” line on the test strip positioned above the liquid level, a sample was allowed to migrate halfway up the strip after which the strip was removed. The results were obtained within 30 min and the strips were evaluated as above.

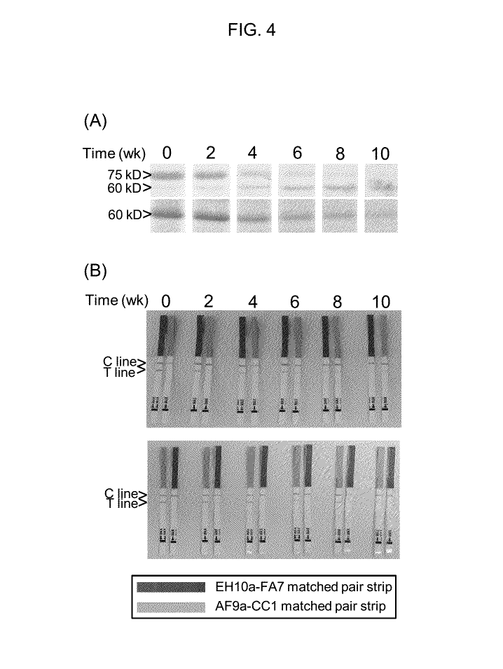

As shown in FIG. 4, the results indicate that the monoclonal antibody match pairs EH10a-FA7 and AF9a-CC1 were able to detect a prominent protein band of 75 kD and 60 kD, respectively. This difference in size may be attributed to a larger-sized glycosylated phytase detected by match pairs EH10a-FA7 from the (yeast-expressed) recombinant phytase. Over time, the detection of the 75 kD protein by monoclonal antibody match pair EH10a-FA7 decreased and its ability to detect the 60 kD increased. In contrast, the ability of the monoclonal antibody match pair AF9a-CC1 to detect the 60 kD protein decreased over time from the GM corn phytase. This suggests that the anti-phytase antibodies can be used to distinguish a (yeast-expressed) glycosylated recombinant phytase (using the EH10a-FA7 match pair) from a lesser glycosylated GM corn phytase (using the AF9a-CC1 match pair). Further, this provides evidence that the epitope binding sites for monoclonal antibodies, EH10a and FA7, to phytase may be glycosylated.

What is claimed is:

1. Monoclonal antibodies that react specifically with Aspergillus niger (phyA2) phytase or a phytase derived from A. niger (phyA2) phytase.

2. The monoclonal antibodies of claim 1 which is produced by hybridoma cell lines EH10a, FA7, AF9a and CC1.

3. The monoclonal antibodies of claim 1 wherein the phytase is found in genetically modified organisms.

4. The monoclonal antibodies of claim 1 wherein the phytase maybe glycosylated.

5. The monoclonal antibodies of claim 1 wherein the phytase may not be glycosylated.

6. A sample in the presence of the monoclonal antibodies of claim 1 which immunologically recognizes the phytase in the sample such that a primary antibody-phytase complex is formed.

7. The antibodies of claim 4 and 5 which may be labelled with gold colloid.

8. A solid support to which the antibodies of claim 1 has been attached.

9. Hybridoma cell lines which produce the monoclonal antibodies of claim 1.

10. The hybridoma cell lines of claim 9 wherein the cell line EH10a, FA7, AF9a and CC1.

11. An immunoassay for the detection of an A. niger (phyA2) phytase or a phytase derived from A. niger (phyA2) phytase in a sample comprising of:
a) employing a solid support upon which the monoclonal antibody of claim 1 is bound to the solid support;
b) incubating the monoclonal antibody of claim 1 on the solid support with a phytase-containing sample which is recognized by the monoclonal antibody;
c) incubating with a secondary anti-phytase antibody which also recognizes the phytase-containing sample to form a monoclonal anti-phytase antibody-phytase sample-secondary anti-phytase antibody complex;
d) measuring the amount of bound anti-phytase antibody as an indication of phytase present;
e) the immunoassay of claim 11 which is an EIA.

12. The immunoassay of claim 11, wherein the solid phase format is composed of multiple stacks and contiguous layers wherein layers are capable of capturing a different phytase from a sample.

13. The sample of claim 12 can be from genetically modified products, such as genetically modified phytase corn.

14. An immunoassay for the detection of an A. niger (phyA2) phytase or a phytase derived from A. niger (phyA2) phytase in a sample comprising of the steps of:
a) preparing a sample in the presence of a primary monoclonal antibody of claim 1 which immunologically recognizes the phytase in the sample such that a primary antibody-phytase complex is form;
b) preparing a solid support format having a measurement in three dimensions to form a volume containing interstitial spaces upon which binding of the solid capture membrane format a secondary antibody capable of immunologically recognizing the phytase and wherein the secondary antibody is conjugated to a means of detection and wherein the secondary antibody also immunologically recognizes the phytase;
c) combining the sample of step (a) with the prepared format of step (b) whereby the sample is drawn through the interstitial spaces of the prepared solid capture membrane format capturing the primary antibody-phytase complex;
d) detecting the phytase by the presence of said capture primary antibody-phytase complex.

15. The immunoassay of claim 14 wherein the solid capture membrane format is polyvinylidene difluoride, nitrocellulose, cellulose acetate, cellulose or nylon.

16. The immunoassay of claim 15, further comprising a sample absorption pad of the solid support format.

17. The immunoassay of claim 16, further comprising a wicking pad of the solid support format.

18. The immunoassay of claim 17 further comprising a strip comprising a labelled anti-phytase antibody.

19. The immunoassay of claim 18 wherein the means of detection is colloidal gold.

20. The immunoassay of claim 19 wherein the phytase is found in genetically modified phytase crops.
Methods for the detection of phytase protein in genetically modified organisms will be obvious to those skilled in the art. The present invention has been described with reference to specific embodiments; it should be made aware that variations and further embodiments are possible and all such variations and embodiments are to be regarded as being within the scope of the present invention.

21-40. (canceled)
